(19) United States
(12) Patent Application Publication
(10) Pub. No.: US 2023/0310298 A1
(43) Pub. Date: Oct. 5, 2023

(54) SKIN CARE FORMULATION

(71) Applicants: Dow Global Technologies LLC, Midland, MI (US); Dow Silicones Corporation, Midland, MI (US); Rohm and Haas Company, Collegeville, PA (US)

(72) Inventors: Wenjun Xu, Eagleville, PA (US); Marc-Andre Courtemanche, Midland, MI (US); Isabelle Van Reeth, Incourt Walloon Brabant (BE); Helene Dihang, Taisnières-sur-Hon (FR); Tian Lan, Langhorne, PA (US); Myoungbae Lee, Midland, MI (US); Matthew Mclaughlin, Midland, MI (US)

(21) Appl. No.: 18/023,155

(22) PCT Filed: Sep. 21, 2021
(86) PCT No.: PCT/US2021/051168

§ 371 Date: Feb. 24, 2023

Related U.S. Application Data
(60) Provisional application No. 63/082,768, filed on Sep. 24, 2020.

(51) Int. Cl. A61K 8/58; A61K 8/73; A61K 8/29; A61K 8/31; A61Q 17/00

(52) U.S. Cl. CPC A61K 8/585; A61K 8/731; A61K 8/29; A61K 8/31; A61Q 17/00; A61K 2800/43; A61K 2800/48

(57) ABSTRACT

An oil/polymer blend is provided comprising: an oil, wherein the oil is selected from the group consisting of silicone fluids, fluids containing 2 to 30 carbon atoms per molecule, and mixtures thereof; and a thickening polymer powder, thickening polymer, wherein the thickening polymer is a silylated cellulose polymer, comprising a cellulose polymer functionalized with —Si(R¹)₃ groups; wherein each R¹ is independently selected from the group consisting of a hydrogen, a C₁₋₈ alkyl group, a C₁₋₈ haloalkyl group, an aryl group, a C₁₋₈ alkylaryl group and a C₁₋₈ haloalkylaryl group; and wherein the cellulose polymer has a weight average molecular weight of 50,000 to 1,500,000 Daltons.

The present invention relates to an oil/polymer blend. In particular, the present invention relates to a skin care formulation comprising an oil/polymer blend comprising an oil, wherein the oil is selected from the group consisting of silicone fluids, fluids containing 2 to 30 carbon atoms per molecule, and mixtures thereof; a thickening polymer powder, thickening polymer, wherein the thickening polymer is a silylated cellulose polymer, comprising a cellulose polymer functionalized with —Si(R¹)₃ groups; wherein each R¹ is independently selected from the group consisting of a hydrogen, a C₁₋₈ alkyl group, a C₁₋₈ haloalkyl group, an aryl group, a C₁₋₈ alkylaryl group and a C₁₋₈ haloalkylaryl group; and wherein the cellulose polymer has a weight average molecular weight of 50,000 to 1,500,000 Daltons.

Consumers increasingly desire skin care formulations that provide long wear properties, such that the formulation might be applied once and last through the work day and beyond without the need for refreshing or touching up. Given todays active lifestyles, it is no simple task to provide such long wear skin care formulations.

An approach to providing such cosmetic formulations is disclosed by Konik et al. in U.S. Pat. No. 6,060,072. In U.S. Pat. No. 6,060,072, Konik et al. disclose water proof or water resistant cosmetic compositions which comprise a styrene-ethylene-propylene copolymer in an amount of 5 to 10%, a combination of a PVP/eicosene copolymer and tricontanyl PVP copolymer in an amount of 0.1 to 50%, a C₈₋₉ isoparaffin, a C₉₋₁₂ aliphatic hydrocarbon, or a combination thereof, in an amount of 50 to 85%.

Notwithstanding, there remains a need for new skin care formulations that provide effective wear resistance with color retention.

The present invention provides an oil/polymer blend comprising: (a) an oil, wherein the oil is selected from the group consisting of silicone fluids, fluids containing 2 to 30 carbon atoms per molecule, and mixtures thereof; and (b) a thickening polymer powder, thickening polymer, wherein the thickening polymer is a silylated cellulose polymer, comprising a cellulose polymer functionalized with —Si(R¹)₃ groups; wherein each R¹ is independently selected from the group consisting of a hydrogen, a C₁₋₈ alkyl group, a C₁₋₈ haloalkyl group, an aryl group, a C₁₋₈ alkylaryl group and a C₁₋₈ haloalkylaryl group; and wherein the cellulose polymer has a weight average molecular weight of 50,000 to 1,500,000 Daltons.

The present invention provides an oil/polymer blend comprising: (a) an oil, wherein the oil is a wherein the cosmetically acceptable oil is selected from the group consisting of linear or branched C₈₋₃₀ alkanes; C₈₋₃₀ alkyl esters; C₈₋₃₀ carboxylic acid esters; C₈₋₃₀ linear or branched alcohols; silicone fluids; and mixtures thereof; and (b) a thickening polymer powder, thickening polymer, wherein the thickening polymer is a silylated cellulose polymer, comprising a cellulose polymer functionalized with —Si(R¹)₃ groups; wherein each R¹ is independently selected from the group consisting of a hydrogen, a C₁₋₈ alkyl group, a C₁₋₈ haloalkyl group, an aryl group, a C₁₋₈ alkylaryl group and a C₁₋₈ haloalkylaryl group; and wherein the cellulose polymer has a weight average molecular weight of 50,000 to 1,500,000 Daltons.

The present invention provides a skin care formulation, comprising: a cosmetically acceptable carrier; a color ingredient; and an oil/polymer blend, wherein the oil/polymer blend comprises: (a) an oil, wherein the oil is a wherein the cosmetically acceptable oil is selected from the group consisting of linear or branched C₈₋₃₀ alkanes; C₈₋₃₀ alkyl esters; C₈₋₃₀ carboxylic acid esters; C₈₋₃₀ linear or branched alcohols; silicone fluids; and mixtures thereof; and (b) a thickening polymer powder, thickening polymer, wherein the thickening polymer is a silylated cellulose polymer, comprising a cellulose polymer functionalized with —Si(R¹)₃ groups; wherein each R¹ is independently selected from the group consisting of a hydrogen, a C₁₋₈ alkyl group, a C₁₋₈ haloalkyl group, an aryl group, a C₁₋₈ alkylaryl group and a C₁₋₈ haloalkylaryl group; and wherein the cellulose polymer has a weight average molecular weight of 50,000 to 1,500,000 Daltons.

The present invention provides a method of enhancing the appearance of skin, comprising: providing a skin care formulation of the present invention, and applying the skin care formulation to skin.

### DETAILED DESCRIPTION

We have now surprisingly found the unique oil/polymer blend, as described herein, when formulated into skin care formulations provide effective thickening (preferably, ≥500 Pa·s at 0.01 sec⁻¹ (more preferably, ≥5,000 Pa·s at 0.01 sec⁻¹)); effective shear thinning (preferably, ≤10 Pa·s at 100 sec⁻¹ (more preferably, ≤0.5 Pa·s at 100 sec⁻¹)); and good rub off resistance, which properties benefit an array of skin care compositions including foundation, lip stick, lip gloss, mascara, hair oils, sunscreen oil and antiperspirant/deodorant formulations.

Unless otherwise indicated, ratios, percentages, parts, and the like are by weight.

As used herein, unless otherwise indicated, the terms “weight average molecular weight” and “M_{w}” are used interchangeably to refer to the weight average molecular weight as measured in a conventional manner with gel permeation chromatography (GPC) and conventional standards, such as polystyrene standards. GPC techniques are discussed in detail in Modem Size Exclusion Liquid Chromatography: Practice of Gel Permeation and Gel Filtration Chromatography, Second Edition, Striegel, et al., John Wiley & Sons, 2009. Weight average molecular weights are reported herein in units of Daltons.

The term “cosmetically acceptable” as used herein and in the appended refers to ingredients typically used in personal care compositions, and is intended to underscore that materials that are toxic when present in the amounts typically found in personal care compositions are not contemplated as part of the present invention.

The term “aesthetic characteristics” as used herein and in the appended claims in reference to an acidic aqueous cleansing formulation refers to visual and tactile sensory properties (e.g., smoothness, tack, lubricity, texture, color, clarity, turbidity, uniformity).

Preferably, the oil/polymer blend of the present invention, comprises: (a) an oil (preferably, 75 to 99.9 wt % (more preferably, 80 to 99.7 wt %; still more preferably, 85 to 99.6 wt %; most preferably, 90 to 99.5 wt %), based on weight of the oil/polymer blend, of the oil), wherein the oil is selected from the group consisting of silicone fluids, fluids containing 2 to 30 carbon atoms (preferably, 5 to 30 carbon atoms; more preferably 8 to 30 carbon atoms; most preferably, 10 to 25 carbon atoms) per molecule, and mixtures thereof; and (b) a thickening polymer (preferably, 0.1 to 25 wt % (more preferably, 0.3 to 20 wt %; still more preferably, 0.4 to 15 wt %; most preferably, 0.5 to 10 wt %), based on weight of the oil/polymer blend, of the thickening polymer), wherein the thickening polymer is a silylated cellulose polymer, comprising a cellulose polymer functionalized with —Si(R¹)₃ groups; wherein the cellulose polymer has a weight average molecular weight of 50,000 to 1,500,000 Daltons; wherein each R¹ is independently selected from the group consisting of a hydrogen, a C₁₋₈ alkyl group, a C₁₋₈ haloalkyl group, an aryl group, a C₁₋₈ alkylaryl group and a C₁₋₈ haloalkylaryl group (preferably, a hydrogen and a C₁₋₈ alkyl group; more preferably, a C₁₋₄ alkyl group; still more preferably, a C₁₋₂ alkyl group; most preferably, a methyl group).

Preferably, the oil/polymer blend of the present invention, comprises 75 to 99.9 wt % (preferably, 80 to 99.7 wt %; more preferably, 85 to 99.6 wt %; most preferably, 90 to 99.5 wt %), based on weight of the oil/polymer blend, of an oil, wherein the oil is selected from the group consisting of silicone fluids, fluids containing 2 to 30 carbon atoms (preferably, 5 to 30 carbon atoms; more preferably 8 to 30 carbon atoms; most preferably, 10 to 25 carbon atoms) per molecule, and mixtures thereof. More preferably, the oil/polymer blend of the present invention, comprises 75 to 99.9 wt % (preferably, 80 to 99.7 wt %; more preferably, 85 to 99.6 wt %; most preferably, 90 to 99.5 wt %), based on weight of the oil/polymer blend, of an oil, wherein the oil is selected from the group consisting of silicone fluids, fluids containing 2 to 30 carbon atoms (preferably, 5 to 30 carbon atoms; more preferably 8 to 30 carbon atoms; most preferably, 10 to 25 carbon atoms) per molecule, and mixtures thereof; wherein the oil is a cosmetically acceptable oil. Still more preferably, the oil/polymer blend of the present invention, comprises 75 to 99.9 wt % (preferably, 80 to 99.7 wt %; more preferably, 85 to 99.6 wt %; most preferably, 90 to 99.5 wt %), based on weight of the oil/polymer blend, of an oil, wherein the oil is a cosmetically acceptable oil selected from the group consisting of linear or branched C₅₋₃₀ alkanes (preferably, C₁₄₋₂₂ mineral oil, isododecane, C₁₁/C₁₃ linear alkane blends (e.g., Cetiol Ultimate), hemisqualane; more preferably, isododecane, C₁₁/C₁₃ linear alkane blends (e.g., Cetiol Ultimate), most preferably, isododecane); C₅₋₃₀ alkyl esters (preferably, 2-methylundecanoate, C₁₂ alkyl benzoate, ethyl decanoate, ethyl dodecanoate, pentaerythrityl tetraethylhexanoate, octyldodecyl neopentanoate, tricaprylin, ethylhexyl isononanoate, methylheptyl isostearate, dicaprylyl maleate, ethylhexyl hydroxystearate); C₂₋₃₀ carboxylic acid esters (preferably, oleic acid, decanoic acid, octanoic acid, linoleic acid, palmitic acid, stearic acid, lauric acid, myristic acid, ricinoleic acid); C₂₋₃₀ linear or branched alcohols (preferably, 2-butyl-1-octanol, cetyl alcohol (aka 1-hexadecanol), stearyl alcohol, dodecanol (aka lauryl alcohol), octanol (aka capryl alcohol), 1-nonanol, 1-decanol (aka capric alcohol), 1-undecanol, 1-tridecanol, myristyl alcohol (aka 1-tetradecanol), 1-pentadecanol, cis-9-hexadecen-1-ol, heptadecyl alcohol, oleyl alcohol (aka 1-octadecenol), 1-nonadecanol, 1-eicosanol, 1-heneicosanol, behenyl alcohol (aka 1-docosanol), cis-13-docosen-1-ol, 1-tetracosanol, ceryl alcohol (aka 1-hexacosanol), 1-heptacosanol, montanyl alcohol, 1-nonacosanol, myricyl alcohol); silicone fluids (preferably, caprylyl methicone, methylhexyl trisiloxane, phenyl trimethicone, cyclosiloxanes (e.g., cyclopentasiloxane, cyclohexasiloxane), polydimethylsiloxane (e.g., 5 cSt 200 fluid)); and mixtures thereof. Yet more preferably, the oil/polymer blend of the present invention, comprises 75 to 99.9 wt % (preferably, 80 to 99.7 wt %; more preferably, 85 to 99.6 wt %; most preferably, 90 to 99.5 wt %), based on weight of the oil/polymer blend, of an oil, wherein the oil is a cosmetically acceptable oil selected from the group consisting of C₁₄₋₂₂ mineral oil, isododecane, C₁₁/C₁₃ linear alkane blend, hemisqualane, 2-methylundecanoate, C₁₂ alkyl benzoate, ethyl decanoate, ethyl dodecanoate, pentaerythrityl tetraethylhexanoate, octyldodecyl neopentanoate, tricaprylin, ethylhexyl isononanoate, methylheptyl isostearate, dicaprylyl maleate, ethylhexyl hydroxystearate, oleic acid, decanoic acid, octanoic acid, linoleic acid, palmitic acid, stearic acid, lauric acid, myristic acid, ricinoleic acid, 2-butyl-1-octanol, cetyl alcohol, stearyl alcohol, dodecanol, octanol, 1-nonanol, 1-decanol, 1-undecanol, 1-tridecanol, myristyl alcohol, 1-pentadecanol, cis-9-hexadecen-1-ol, heptadecyl alcohol, oleyl alcohol, 1-nonadecanol, 1-eicosanol, 1-heneicosanol, behenyl alcohol, cis-13-docosen-1-ol, 1-tetracosanol, ceryl alcohol, 1-heptacosanol, montanyl alcohol, 1-nonacosanol, myricyl alcohol, caprylyl methicone, methylhexyl trisiloxane, phenyl trimethicone, cyclopentasiloxane, cyclohexasiloxane, polydimethylsiloxane and mixtures thereof. Most preferably, the oil/polymer blend of the present invention, comprises 75 to 99.9 wt % (preferably, 80 to 99.7 wt %; more preferably, 85 to 99.6 wt %; most preferably, 90 to 99.5 wt %), based on weight of the oil/polymer blend, of an oil, wherein the oil is a cosmetically acceptable oil selected from the group consisting of isododecane, caprylyl methicone and mixtures thereof.

Preferably, the oil/polymer blend of the present invention, comprises 0.1 to 25 wt % (preferably, 1 to 20 wt %; more preferably, 2 to 15 wt %; most preferably, 2.5 to 7.5 wt %), based on weight of the oil/polymer blend, of the thickening polymer; wherein the thickening polymer is a silylated cellulose polymer; wherein the silylated cellulose polymer comprises a cellulose polymer functionalized with —Si(R¹)₃ groups; wherein the cellulose polymer has a weight average molecular weight of 50,000 to 1,500,000 Daltons (preferably, 50,000 to 1,250,000 Daltons; more preferably, 80,000 to 1,150,000 Daltons; most preferably, 100,000 to 1,100,000 Daltons); wherein each R¹ is independently selected from the group consisting of a hydrogen, a C₁₋₈ alkyl group, a C₁₋₈ haloalkyl group, an aryl group, a C₁₋₈ alkylaryl group and a C₁₋₈ haloalkylaryl group. More preferably, the oil/polymer blend of the present invention, comprises 0.1 to 25 wt % (preferably, 1 to 20 wt %; more preferably, 2 to 15 wt %; most preferably, 2.5 to 7.5 wt %), based on weight of the oil/polymer blend, of a thickening polymer, wherein the thickening polymer is a silylated cellulose polymer; wherein the silylated cellulose polymer comprises a cellulose polymer functionalized with —Si(R¹)₃ groups; wherein the cellulose polymer has a weight average molecular weight of 50,000 to 1,500,000 Daltons (preferably, 50,000 to 1,250,000 Daltons; more preferably, 80,000 to 1,150,000 Daltons; most preferably, 100,000 to 1,100,000 Daltons); wherein each R¹ is independently selected from the group consisting of a hydrogen and a C₁₋₈ alkyl group. Still more preferably, the oil/polymer blend of the present invention, comprises 0.1 to 25 wt % (preferably, 1 to 20 wt %; more preferably, 2 to 15 wt %; most preferably, 2.5 to 7.5 wt %), based on weight of the oil/polymer blend, of a thickening polymer, wherein the thickening polymer is a silylated cellulose polymer; wherein the silylated cellulose polymer comprises a cellulose polymer functionalized with —Si(R¹)₃ groups; wherein the cellulose polymer has a weight average molecular weight of 50,000 to 1,500,000 Daltons (preferably, 50,000 to 1,250,000 Daltons; more preferably, 80,000 to 1,150,000 Daltons; most preferably, 100,000 to 1,100,000 Daltons); wherein each R¹ is independently selected from the group consisting of a C₁₋₄ alkyl group. Yet still more preferably, the oil/polymer blend of the present invention, comprises 0.1 to 25 wt % (preferably, 1 to 20 wt %; more preferably, 2 to 15 wt %; most preferably, 2.5 to 7.5 wt %), based on weight of the oil/polymer blend, of a thickening polymer, wherein the thickening polymer is a silylated cellulose polymer; wherein the silylated cellulose polymer comprises a cellulose polymer functionalized with —Si(R¹)₃ groups; wherein the cellulose polymer has a weight average molecular weight of 50,000 to 1,500,000 Daltons (preferably, 50,000 to 1,250,000 Daltons; more preferably, 80,000 to 1,150,000 Daltons; most preferably, 100,000 to 1,100,000 Daltons); wherein each R¹ is independently selected from the group consisting of a C₁₋₂ alkyl group. Most preferably, the oil/polymer blend of the present invention, comprises 0.1 to 25 wt % (preferably, 1 to 20 wt %; more preferably, 2 to 15 wt %; most preferably, 2.5 to 7.5 wt %), based on weight of the oil/polymer blend, of a thickening polymer, wherein the thickening polymer is a silylated cellulose polymer; wherein the silylated cellulose polymer comprises a cellulose polymer functionalized with —Si(R¹)₃ groups; wherein the cellulose polymer has a weight average molecular weight of 50,000 to 1,500,000 Daltons (preferably, 50,000 to 1,250,000 Daltons; more preferably, 80,000 to 1,150,000 Daltons; most preferably, 100,000 to 1,100,000 Daltons); wherein each R¹ is a methyl group.

Preferably, the oil/polymer blend of the present invention, comprises 0.1 to 25 wt % (preferably, 1 to 20 wt %; more preferably, 2 to 15 wt %; most preferably, 2.5 to 7.5 wt %), based on weight of the oil/polymer blend, of a thickening polymer, wherein the thickening polymer is a silylated cellulose polymer of formula I

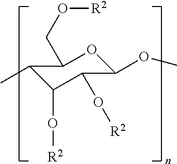

wherein n is an average of 308 to 9,252 (preferably, 308 to 6,167; more preferably, 3083 to 4,317); wherein each R² is independently selected from the group consisting of a hydrogen, a C₁₋₈ alkyl group and a —Si(R¹)₃ group (preferably, a hydrogen, a C₁₋₄ alkyl group and a —Si(R¹)₃ group; more preferably, a hydrogen, a C₁₋₂ alkyl group and a —Si(R²)₃ group; most preferably, a hydrogen and a —Si(R¹)₃ group); wherein each R¹ is independently selected from the group consisting of a hydrogen, a C₁₋₈ alkyl group, a C₁₋₈ haloalkyl group, an aryl group, a C₁₋₈ alkylaryl group and a C₁₋₈ haloalkylaryl group (preferably, a hydrogen and a C₁₋₈ alkyl group; more preferably, a C₁₋₄ alkyl group; still more preferably, a C₁₋₂ alkyl group; most preferably, a methyl group).

Preferably, the thickening polymer is a silylated cellulose polymer; wherein the silylated cellulose polymer comprises a cellulose polymer functionalized with —Si(R¹)₃ groups having a degree of substitution, DS, of the —Si(R¹)₃ groups of 1.4 to 3.0 (preferably, 1.5 to 3.0; more preferably, 2.0 to 3.0; most preferably, 2.25 to 3.0) determined by Attenuated Total Reflection-Fourier Transform Infrared (ATR-FTIR) Spectroscopy.

Preferably, the skin care formulation of the present invention comprises: a cosmetically acceptable carrier (preferably, 30 to 92 wt % (more preferably, 35 to 92 wt %; most preferably, 40 to 80 wt %), based on weight of the skin care formulation, of the cosmetically acceptable carrier); a color ingredient (preferably, 0.01 to 25 wt % (more preferably, 0.1 to 20 wt %; most preferably, 1 to 10 wt %), based on weight of the skin care formulation, of the color ingredient); and an oil/polymer blend (preferably, 5 to 69.9 wt % (more preferably, 7 to 50 wt %; most preferably, 15 to 30 wt %), based on weight of the skin care formulation, of the oil/polymer blend); wherein the oil/polymer blend, comprises: (a) an oil (preferably, 75 to 99.9 wt % (more preferably, 80 to 99.7 wt %; still more preferably, 85 to 99.6 wt %; most preferably, 90 to 99.5 wt %), based on weight of the oil/polymer blend, of the oil), wherein the oil is a cosmetically acceptable oil from the group consisting of linear or branched C₈₋₃₀ alkanes (preferably, C₁₄₋₂₂ mineral oil, isododecane, C₁₁/C₁₃ linear alkane blends (e.g., Cetiol Ultimate), hemisqualane; more preferably, isododecane, C₁₁/C₁₃ linear alkane blends (e.g., Cetiol Ultimate), most preferably, isododecane); C₈₋₃₀ alkyl esters (preferably, 2-methylundecanoate, C₁₂ alkyl benzoate, ethyl decanoate, ethyl dodecanoate, pentaerythrityl tetraethylhexanoate, octyldodecyl neopentanoate, tricaprylin, ethylhexyl isononanoate, methylheptyl isostearate, dicaprylyl maleate, ethylhexyl hydroxystearate); C₈₋₃₀ carboxylic acid esters (preferably, e.g., oleic acid, decanoic acid, octanoic acid, linoleic acid, palmitic acid, stearic acid, lauric acid, myristic acid, ricinoleic acid); C₈₋₃₀ linear or branched alcohols (preferably, 2-butyl-1-octanol, cetyl alcohol (aka 1-hexadecanol), stearyl alcohol, dodecanol (aka lauryl alcohol), octanol (aka capryl alcohol), 1-nonanol, 1-decanol (aka capric alcohol), 1-undecanol, 1-tridecanol, myristyl alcohol (aka 1-tetradecanol), 1-pentadecanol, cis-9-hexadecen-1-ol, heptadecyl alcohol, oleyl alcohol (aka 1-octadecenol), 1-nonadecanol, 1-eicosanol, 1-heneicosanol, behenyl alcohol (aka 1-docosanol), cis-13-docosen-1-ol, 1-tetracosanol, ceryl alcohol (aka 1-hexacosanol), 1-heptacosanol, montanyl alcohol, 1-nonacosanol, myricyl alcohol); silicone fluids (preferably, caprylyl methicone methylhexyl trisiloxane, phenyl trimethicone, polydimethylsiloxane (e.g., 5 cSt 200 fluid)); and mixtures thereof; and (b) a thickening polymer (preferably, 0.1 to 25 wt % (more preferably, 1 to 20 wt %; still more preferably, 2 to 15 wt %; most preferably, 2.5 to 7.5 wt %), based on weight of the oil/polymer blend, of the thickening polymer), wherein the thickening polymer is a silylated cellulose polymer, comprising a cellulose polymer functionalized with —Si(R¹)₃ groups; wherein each R¹ is independently selected from the group consisting of a hydrogen, a C₁₋₈ alkyl group, a C₁₋₈ haloalkyl group, an aryl group, a C₁₋₈ alkylaryl group and a C₁₋₈ haloalkylaryl group (preferably, a hydrogen and a C₁₋₈ alkyl group; more preferably, a C₁₋₄ alkyl group; still more preferably, a C₁₋₂ alkyl group; most preferably, a methyl group).

Preferably, the skin care formulation of the present invention, comprises 30 to 92 wt % (preferably, 35 to 92 wt %; more preferably, 40 to 80 wt %), based on weight of the skin care formulation, of a cosmetically acceptable carrier. More preferably, the skin care formulation of the present invention, comprises 30 to 92 wt % (preferably, 35 to 92 wt %; more preferably, 40 to 80 wt %), based on weight of the skin care formulation, of a cosmetically acceptable carrier; wherein the color ingredient and the oil/polymer blend are dispersed in the cosmetically acceptable carrier. Still more preferably, the skin care formulation of the present invention, comprises 30 to 92 wt % (preferably, 35 to 92 wt %; more preferably, 40 to 80 wt %), based on weight of the skin care formulation, of a cosmetically acceptable carrier; wherein the color ingredient and the oil/polymer blend are dispersed in the cosmetically acceptable carrier. Most preferably, the skin care formulation of the present invention, comprises 30 to 92 wt % (preferably, 35 to 92 wt %; more preferably, 40 to 80 wt %), based on weight of the skin care formulation, of a cosmetically acceptable carrier; wherein the color ingredient and the oil/polymer blend are dispersed in the cosmetically acceptable carrier.

Preferably, the skin care formulation of the present invention, comprises 30 to 92 wt % (preferably, 35 to 92 wt %; more preferably, 40 to 80 wt %), based on weight of the skin care formulation, of a cosmetically acceptable carrier; wherein the cosmetically acceptable carrier is selected to be capable of evaporating upon application of the skin care formulation to mammalian skin (preferably, human skin).

Preferably, the skin care formulation of the present invention, comprises 30 to 92 wt % (preferably, 35 to 92 wt %; more preferably, 40 to 80 wt %), based on weight of the skin care formulation, of a cosmetically acceptable carrier; wherein the cosmetically acceptable is selected from the group consisting of water (e.g., deionized, distilled water); short chain alcohols (e.g., C₁₄ straight or branched chain alcohols such as methyl alcohol, ethyl alcohol, propyl alcohol, isopropyl alcohol, butyl alcohol); glycols (e.g., ethylene glycol, propylene glycol, butylene glycol, pentylene glycol, hexylene glycol, dipropylene glycol, ethoxydiglycol); cyclosiloxanes (e.g., cyclopentasiloxane, cyclohexasiloxane); glycerin; isododecane; isohexadecane; acetone; methyl acetate; butyl cellosolve and mixtures thereof.

More preferably, the skin care formulation of the present invention, comprises 30 to 92 wt % (preferably, 35 to 92 wt %; more preferably, 40 to 80 wt %), based on weight of the skin care formulation, of a cosmetically acceptable carrier; wherein the cosmetically acceptable carrier includes at least one of water and isododecane (preferably, at least one of deionized water, distilled water and isododecane; more preferably, at lease one of deionized water and isododecane).

Preferably, the skin care formulation of the present invention comprises a color ingredient. More preferably, the skin care formulation of the present invention comprises a color ingredient; wherein the color ingredient is selected from the group consisting of inorganic pigments, organic pigments, dyes, aqueous pigment dispersions and mixtures thereof. Still more preferably, the skin care formulation of the present invention comprises a color ingredient; wherein the color ingredient is selected from the group consisting of Ext. D&C Yellow No. 2, Ext. D & C Violet No. 2, FD&C Red No. 4, FD&C Red No. 40, FD&C Yellow No. 5, FD&C Yellow No. 6, FD&C Green No. 3, FD&C Blue No. 1, D&C Yellow No. 7, D&C Yellow No. 8, D&C Yellow No. 10, D&C Yellow No. 11, D&C Violet No. 2, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 34, D&C Red No. 33, D&C Red No. 36, D&C Green No. 5, D&C Green No. 6, D&C Green No. 8, D&C Blue No. 4, D&C Orange No. 4, D&C Orange No. 5, D&C Orange No. 10, D&C Orange No. 11, D&C Brown No. 1, Aluminum powder, Annatto, Bismuth citrate, Bismuth Oxychloride, Bronze powder, Caramel, Carmine, β-Carotene, Chromium hydroxide green, Chromium oxide green, Copper chlorophyllin, Copper powder, Dihydroxyacetone, Ferric Ammonium ferrocyanide, Ferric ferrocyanide, Guanine, Iron oxide, Manganese Violet, Mica, Silver, Titanium Dioxide, Ultramarine, Zinc Oxide and mixtures thereof. Still more preferably, the skin care formulation of the present invention comprises a color ingredient; wherein the color ingredient includes at least one iron oxide and titanium dioxide. Most preferably, preferably, the skin care formulation of the present invention comprises a color ingredient; wherein the color ingredient includes a mixture of iron oxides and titanium dioxide.

Preferably, the skin care formulation of the present invention, optionally, further comprises an optional additive. More preferably, the skin care formulation of the present invention, further comprises an optional additive, wherein the optional additive is selected from the group consisting of suncare actives, film formers, emollients, preservatives, antioxidants, fragrances, humectants, rheology modifiers, aesthetic modifiers, vitamins, skin protectants, oils, emulsifiers, surfactants, pearlizers, consistency factors, thickeners, super fatting agents, stabilizers, polymers, silicone compounds, fats, waxes, lecithins, phospholipids, fillers, light management powders and particles, and mixtures thereof.

Preferably, the skin care formulation of the present invention, further comprises a suncare active. More preferably, the skin care formulation of the present invention further comprises a suncare active; wherein the suncare active is a UV radiation absorbing agent. Still more preferably, the skin care formulation of the present invention, further comprises a suncare active, wherein the suncare active is a UV radiation absorbing agent selected from the group consisting of physical blockers (e.g., red petrolatum, titanium dioxide, zinc oxide) and chemical absorbers (e.g., 1-(4-methoxyphenol)-3-(4-tert-butylphenyl)propane-1,3-dione (INCI: Butyl Methoxydibenzoylmethane); 2-hydroxy-4-methoxybenzophenone (INCI: Benzophenone-3); dioxybenzone; sulisobenzone; menthyl anthranilate; para-aminobenzoic acid; amyl paradimethylaminobenzoic acid; octyl para-dimethylaminobenzoate; ethyl 4-bis (hydroxypropyl) para-aminobenzoate; polyethylene glycol (PEG-25) para-aminobenzoate; ethyl 4-bis (hydroxypropyl) aminobenzoate; diethanolamine para-methyoxycinnamate; 2-ethoxyethyl para-methoxycinnamate; ethylhexyl para-methoxycinnamate; octyl para-methoxycinnamate; isoamyl para-methoxycinnamate; 2-ethylhexyl-2-cyano-3,3-diphenyl-acrylate; 2-ethylhexyl-2-cyano-3,3-diphenyl-2-propenoate (INCI: octocrylene); 2-ethylhexyl-2-hydroxybenzoate (INCI: Ethylhexyl Salicylate); homomenthyl salicylate; glyceryl aminobenzoate; triethanolamine salicylate; digalloyl trioleate; lawsone with dihydroxyacetone; 2-phenylbenzimidazole-5-sulfonic acid; 4-methylbenzylidine camphor; avobenzone; triazines; benzotriazoles; vinyl group-containing amides; cinnamic acid amides; sulfonated benzimidazoles); 3,3,5-trimethylcyclohexyl 2-hydroxybenzoate (INCI: Homosalate). Yet more preferably, the skin care formulation of the present invention, further comprises of a suncare active, wherein the suncare active is a UV radiation absorbing agent comprises a mixture of UV radiation absorbing agents. Most preferably, the skin care formulation of the present invention, comprises a suncare active, wherein the suncare active is a UV radiation absorbing agent is a mixture of UV absorbing agents including at least one of titanium dioxide; 1-(4-methoxyphenol)-3-(4-tert-butylphenyl)propane-1,3-dione; 2-ethylhexyl-2-hydroxybenzoate; 2-ethyhexyl-2-cyano-3,3-diphenyl-2-propenoate; 2-hydroxy-4-methoxybenzophenone and 3,3,5-trimethylcyclohexyl 2-hydroxybenzoate.

Preferably, the skin care formulation of the present invention has a pH of 4 to 9. More preferably, the skin care formulation of the present invention has a pH of 4.5 to 8.5. Still more preferably, the skin care formulation of the present invention has a pH of 5.0 to 8.0. Most preferably, the skin care formulation of the present invention has a pH of 5.5 to 7.5.

Preferably, the skin care formulation of the present invention is provided a product form selected from the group consisting of a cream, an aqueous solution, an emulsion (e.g., water-in-oil emulsion, oil-in-water emulsion), an oil, an ointment, a paste, a gel, a lotion, a milk, a foam, a stick and a suspension.

The skin care formulation of the present invention are useful for enhancing the appearance of skin through application to the skin. Preferably, the skin care formulation of the present invention applies easily to the skin, leaving a clear vivid color that remains in place at least through the work day and preferably thereafter.

Some embodiments of the present invention will now be described in detail in the following Examples.

### Examples S1-S25: Synthesis of Thickening Polymer

To a two liter three-neck flask equipped with a nitrogen inlet and a temperature controller was weighed the type and amount of cellulose noted in TABLE 1. Then N,N-dimethylacetamide (DMAc) solvent was added to the flask in the amount noted in TABLE 1. The flask contents were then placed under an atmosphere of nitrogen. Then hexamethyldisilazane (HMDS) was added to the flask contents all at once in the amount noted in TABLE 1. The flask contents were then slowly heated while stirring to the set temperature, T1, noted in TABLE 1 and stirred for time, t¹, in hours noted in TABLE 1. The heat source was then removed and the flask contents were left to cool. Then xylene (Xyl) was added to the flask contents in the amount noted in TABLE 1 and the flask contents were then heated to set temperature, T₂, noted in TABLE 1 and stirred for time, t², in hours noted in TABLE 1. The product solution was then transferred to a separatory funnel and subjected to non-solvent precipitation by dropwise addition into two liters of vigorously stirring methanol. The product was precipitated in the form of a thread or flakes. The product was isolated by filtration, and the product was dried in a vacuum oven at 50° C. overnight. The product was then suspended in 500 mL of methanol and left overnight, then refiltered and dried in vacuum oven at 50° C. overnight.

TABLE 1

| | Cellulose | | DMAc | HMDS | T₁ | t¹ | Xyl | T₂ | t² |
|---|---|---|---|---|---|---|---|---|---|
| Example | Type | (g) | (g) | (g) | (° C.) | (hr) | (g) | (° C.) | (hr) |
| S1 | A | 15.2 | 300 | 40.0 | 140 | 3.0 | 400 | 140 | 4.0 |
| S2 | B | 15.2 | 330 | 50.0 | 140 | 2.0 | 320 | 140 | 4.0 |
| S3 | B | 15.2 | 400 | 50.0 | 140 | 2.0 | 600 | 140 | 4.0 |
| S4 | A | 15.0 | 300 | 50.0 | 130 | 5.0 | 300 | 140 | 6.0 |
| S5 | C | 15.1 | 330 | 49.9 | 130 | 7.5 | 400 | 125 | 5.0 |
| S6 | D | 15.0 | 331 | 49.8 | 130 | 7.5 | 400 | 125 | 5.0 |
| S7 | E | 15.0 | 330 | 50.0 | 130 | 5.0 | 400 | 140 | 6.0 |
| S8 | C | 15.2 | 333 | 149.9 | 135 | 3.5 | 400 | 135 | 5.0 |
| S9 | D | 15.1 | 332 | 150.1 | 135 | 3.5 | 400 | 135 | 5.0 |
| S10 | F | 15.5 | 329 | 49.7 | 135 | 5.0 | 400 | 120 | 3.0 |
| S11 | G | 15.4 | 329 | 50.0 | 135 | 5.0 | 400 | 120 | 3.0 |
| S12 | C | 2.0 | 39 | 5.96 | 130 | 4.0 | 0 | — | — |
| S13 | H | 15.2 | 337 | 50.1 | 135 | 6.0 | 400 | 135 | 3.5 |
| S14 | G | 15.2 | 315 | 49.7 | 135 | 4.0 | 400 | 130 | 3.0 |
| S15 | G | 15.2 | 308 | 50.2 | 135 | 4.0 | 400 | 130 | 3.0 |
| S16 | G | 15.2 | 315 | 98.7 | 80 | 4.0 | 300 | 130 | 5.5 |
| S17 | G | 15.3 | 308 | 99.0 | 80 | 4.0 | 320 | 130 | 5.5 |
| S18 | F | 15.1 | 312 | 50.0 | 130 | 5.0 | 400 | 105 | 8.0 |
| S19 | G | 15.2 | 324 | 50.2 | 130 | 5.0 | 400 | 105 | 8.0 |
| S20 | E | 15.2 | 318 | 49.9 | 130 | 4.0 | 330 | 130 | 5.0 |
| S21 | C | 15.0 | 328 | 49.6 | 130 | 4.0 | 330 | 130 | 5.0 |
| S22 | G | 15.2 | 329 | 19.9 | 130 | 4.0 | 330 | 120 | 2.0 |
| S23 | G | 15.2 | 326 | 30.0 | 130 | 4.0 | 330 | 120 | 2.0 |
| S24 | A | 15.2 | 327 | 49.9 | 130 | 4.0 | 330 | 130 | 4.0 |
| S25 | A | 15.2 | 378 | 49.9 | 130 | 4.0 | 330 | 130 | 4.0 |

A Avicel PH-101 (51 μm) microcrystalline cellulose having weight average molecular weight of 60,000 Daltons available from Sigma-Aldrich
B Crystal PL HD eucalyptus pulp having weight average molecular weight of 400,000 Daltons available from Bahi Specialty Cellulose
C Biofloc 92 MV wood pulp having weight average molecular weight of 600,000 Daltons available from Tartas (Rayonier)
D Biofloc XV wood pulp having weight average molecular weight of 900,000 Daltons available from Tartas (Rayonier)
E CS-2400 cotton linters having weight average molecular weight of 1,000,000 Daltons available from Gaomi
F E4 wood pulp having weight average molecular weight of 100,000 Daltons available from GP Cellulose
G E60-HB wood pulp having weight average molecular weight of 125,000 Daltons available from GP Cellulose
H Biofloc 94 MV wood pulp having weight average molecular weight of 600,000 Daltons available from Tartas (Rayonier)

### DS Determination

The degree of substitution, DS, of —Si(CH₃)₃ in the thickening polymers prepared according to Examples S1-S25 was determined using well known techniques based on Attenuated Total Reflection-Fourier Transform Infrared Spectroscopy analyzing spectra peak areas calculated with MATLAB using the spectral parameters provided in TABLE 2 with the DS values determined as reported in TABLE 3.

TABLE 2

| | |
|---|---|
| Integration (cm⁻¹) | Baseline (cm⁻¹) |

| Species | Start | End | Start | End |
|---|---|---|---|---|
| —Si(CH₃)₃ | 1277 | 1220 | 1278 | 1219 |
| —OH | 3685 | 3033 | 3697 | 3040 |

TABLE 3

| | IR Peak Area | | | | Si | OH |
|---|---|---|---|---|---|---|
| Example | Si(CH₃)₃ | CO | OH | DS | (wt %) | (wt %) |
| S2 | 1.28 | 9.92 | 0.264 | 2.58 | 20.8 | 2.04 |
| S3 | 1.47 | 12.0 | 0.686 | 2.20 | 19.2 | 4.26 |
| S4 | 1.44 | 13.3 | 1.59 | 1.61 | 16.2 | 8.49 |
| S5 | 0.963 | 7.35 | 0.426 | 2.23 | 19.4 | 4.07 |
| S6 | 1.62 | 12.8 | 0.714 | 2.23 | 19.4 | 4.06 |
| S7 | 0.597 | 4.95 | 0.351 | 2.05 | 18.6 | 5.19 |
| S8 | 0.938 | 7.24 | 0.282 | 2.43 | 20.2 | 2.89 |
| S9 | 0.760 | 6.51 | 0.382 | 2.15 | 19.0 | 4.54 |
| S10 | 1.08 | 8.49 | 0.248 | 2.54 | 20.6 | 2.26 |
| S11 | 1.03 | 7.67 | 0.0194 | 2.96 | 22.1 | 0.20 |
| S12 | 1.13 | 7.84 | −0.0337 | 3.00 | 22.2 | 0 |
| S13 | 0.811 | 8.81 | 1.51 | 1.22 | 13.7 | 12.1 |
| S14 | 1.63 | 12.6 | 0.237 | 2.69 | 21.2 | 1.47 |
| S15 | 2.12 | 15.3 | −0.151 | 3.00 | 22.2 | 0 |
| S16 | 1.34 | 9.58 | −0.00681 | 3.00 | 22.2 | 0 |
| S17 | 0.623 | 4.47 | 0.0535 | 2.81 | 21.6 | 0.88 |
| S18 | 0.933 | 6.84 | 0.102 | 2.76 | 21.4 | 1.11 |
| S19 | 1.08 | 7.82 | 0.215 | 2.60 | 20.8 | 1.97 |
| S20 | 1.33 | 9.27 | −0.0648 | 3.00 | 22.2 | 0 |
| S21 | 0.491 | 3.48 | −0.00282 | 3.00 | 22.2 | 0 |
| S22 | 0.205 | 2.09 | 0.165 | 1.84 | 17.5 | 6.70 |
| S23 | 1.11 | 8.44 | 0.248 | 2.55 | 20.7 | 2.20 |
| S24 | 0.728 | 10.5 | 2.92 | 0.72 | 9.47 | 18.1 |
| S25 | 2.02 | 15.1 | −0.218 | 3.00 | 22.2 | 0 |

### Comparative Example C1 and Examples 1-4: Skin Care Formulations

The skin care formulations of Comparative Example C1 and Examples 1-4 were prepared having the formulation noted in TABLE 4. The Phase B ingredients were mixed in a separate container. The Phase C ingredients were combined in a separate container with mixing until uniform. The Phase A and D ingredients were combined in a separate beaker and mixed until uniform. The mixed Phase C ingredients were then added to the combined Phase A and D ingredients with shear at 450 rpm until homogeneous. Then the mixed Phase B ingredients were then slowly added to the combined Phase A, C and D ingredients with shear at 700 rpm. The combined formulation was then mixed for an additional 5 minutes with shear at 2,000 rpm to provide the product skin care formulation.

TABLE 4

| | | Example | | | | |
|---|---|---|---|---|---|---|
| | | C1 | 1 | 2 | 3 | 4 |
| | | (wt | (wt | (wt | (wt | (wt |
| Phase | Ingredient INCI name | %) | %) | %) | %) | %) |
| A | Lauryl PEG-10 | 6 | 6 | 6 | 6 | 6 |
| | Tris(trimethylsiloxy)silyethyl | | | | | |
| | Dimeticone¹ | | | | | |
| A | Caprylyl methicone² | 4 | 4 | 4 | 4 | 4 |
| A | Isododecane³ | 15.75 | 18 | 18 | 18 | 18 |
| B | Deionized water | 53 | 53 | 53 | 53 | 53 |
| B | Sodium chloride | 1 | 1 | 1 | 1 | 1 |
| B | Phenoxyethanol (and) | 1 | 1 | 1 | 1 | 1 |
| | Ethylhexylglycerin⁴ | | | | | |
| B | Glycerin | 5 | 5 | 5 | 5 | 5 |
| D | Iron Oxide (CI 77499), | 0.07 | 0.07 | 0.07 | 0.07 | 0.07 |
| | dimethicone⁵ | | | | | |
| C | Iron Oxide (CI 77491), | 0.25 | 0.25 | 0.25 | 0.25 | 0.25 |
| | dimethicone⁶ | | | | | |
| C | Iron Oxide (CI 77492), | 1.09 | 1.09 | 1.09 | 1.09 | 1.09 |
| | dimethicone⁷ | | | | | |
| C | Titanium Dioxide (and) | 5.81 | 5.81 | 5.81 | 5.81 | 5.81 |
| | dimethicone⁸ | | | | | |
| C | Caprylyl Methicone² | 3.28 | 3.28 | 3.28 | 3.28 | 3.28 |
| D | Isododecane (and) Acrylates/ | 3.75 | — | — | — | — |
| | Polytimethylsiloxymeth- | | | | | |
| | acrylate Copolymer⁹ | | | | | |
| D | Example S16 | — | 1.5 | — | — | — |
| D | Example S17 | — | — | 1.5 | — | — |
| D | Example S20 | — | — | — | 1.5 | — |
| D | Example S21 | — | — | — | — | 1.5 |

¹Available from The Dow Chemical Company under tradename DOWSIL ™ ES-5300.
²Available from The Dow Chemical Company under tradename DOWSIL ™ FZ-3196.
³Available from The Innovation Company under tradename Creasil ID CG.
⁴Available from Schulke Inc. under tradename Euxyl PE 9010.
⁵Available from Miyoshi America under tradename SAT-B-335198.
⁶Available from Miyoshi America under tradename SAT-Y-338075.
⁷Available from Miyoshi America under tradename SAT-R-338073.
⁸Available from Miyoshi America under tradename SAT-TRI-77891.
⁹Available from The Dow Chemical Company under tradename DOWSIL ™ FA4002 ID.

### Thickening and Shear Thinning

The viscosity of the skin care formulations of Comparative Example C1 and Examples 1-4 was determined using a TA Instruments DHR-3 rheometer at 25° C., equipped with a stainless steel 60 mm, 0.5° cone and plate sensor, a gap set at 17 microns and a shear rate of 6.31 s⁻¹. Shear sweep method was used with shear rate ranges from 0.01 s⁻¹ to 100 s⁻¹. Viscosities measured at low shear (0.1 s⁻¹) and high shear (100 s⁻¹) are reported in TABLE 5.

The shear thinning efficiency of the thickeners was assessed by fitting the viscosity data to the power-law fluid equation

η=K·γⁿ⁻¹

wherein η is the viscosity, γ is the shear rate, K is the flow consistency index and n is the flow behavior index. For a shear thinning fluid, n<1. Smaller n values indicate a higher degree of shear thinning. The calculated shear thinning efficiencies for the thickeners is reported in TABLE 5.

As shown in TABLE 5, data for all of the thickeners fitted well to the power-law fluid equation, with the coefficient of determination, r², value being close to 1. The flow behavior index (n) for the inventive thickeners was much lower than the commercial material (DOWSIL™ FA4002 ID Silicone Acrylate), indicating a much higher degree of sheer thinning which is highly favorable in personal care formulations for better sprayability and spreadability of the formulation. The inventive polymer also lead to a much higher increase in formulation viscosity than the commercial material (DOWSIL™ FA4002 ID Silicone Acrylate), indicating a much higher thickening efficiency for the inventive polymer.

TABLE 5

| | | | | Fitted | Flow |
|---|---|---|---|---|---|
| Skin Care | | Viscosity (Pa · s) | | power | behavior |
| Formulation | Thickener | 0.01 sec⁻¹ | 100 sec⁻¹ | law equation | index, n |
| C1 | Commercial | 3.99 | 0.4 | η = 1.125 · γ^{-0.256} | 0.744 |
| | material¹ | | | r² = 0.967 | |
| 1 | Example S16 | 795 | 2.5 | η = 36.351 · γ^{-0.064} | 0.360 |
| | | | | r² = 0.9947 | |
| 2 | Example S17 | 1.333 | 2.8 | η = 45.241 · γ^{-0.683} | 0.317 |
| | | | | r² = 0.9901 | |
| 3 | Example S20 | 3.276 | 3.9 | η = 79.197 · γ^{-0.736} | 0.264 |
| | | | | r² = 0.9921 | |
| 4 | Example S21 | 2.520 | 2.6 | η = 57.845 · γ^{-0.767} | 0.233 |
| | | | | r² = 0.993 | |

¹DOWSIL ™ FA4002 ID Silicon Isododecane (and) Acrylates/Polytrimethylsiloxymethacrylate Copolymer available from The Dow Chemical Company

### Comparative Example C2-C3 and Examples 5-9: Skin Care Formulations

The skin care formulations of Comparative Examples C2-C3 and Examples 5-9 were prepared having the formulation noted in TABLE 6. The Phase B ingredients were mixed in a separate container. The Phase C ingredients were combined in a separate container with mixing until uniform. The Phase A and D ingredients were combined in a separate beaker and mixed until uniform. The mixed Phase C ingredients were then added to the combined Phase A and D ingredients with shear at 450 rpm until homogeneous. Then the mixed Phase B ingredients were then slowly added to the combined Phase A, C and D ingredients with shear at 700 rpm. The combined formulation was then mixed for an additional 5 minutes with shear at 2,000 rpm to provide the product skin care formulation.

TABLE 6

| | | Example (wt %) | | | | | | |
|---|---|---|---|---|---|---|---|---|
| Phase | Ingredient INCI name | C2 | C3 | 5 | 6 | 7 | 8 | 9 |
| A | Lauryl PEG-10 | 6 | 6 | 6 | 6 | 6 | 6 | 6 |
| | Tris(trimethylsiloxy)silyethyl | | | | | | | |
| | Dimeticone¹ | | | | | | | |
| A | Caprylyl methicone² | 3.6 | 3 | 3 | 3 | 3 | 3 | 3 |
| A | Isododecane³ | 12 | 11.1 | 12 | 12 | 12 | 12 | 12 |
| B | Deionized water | 60.9 | 60.9 | 60.9 | 60.9 | 60.9 | 60.9 | 60.9 |
| B | Sodium chloride | 1 | 1 | 1 | 1 | 1 | 1 | 1 |
| B | Phenoxyethanol (and) | 1 | 1 | 1 | 1 | 1 | 1 | 1 |
| | Ethylhexylglycerin⁴ | | | | | | | |
| B | Glycerin | 5 | 5 | 5 | 5 | 5 | 5 | 5 |
| D | Iron Oxide (CI 77499), | 0.07 | 0.07 | 0.07 | 0.07 | 0.07 | 0.07 | 0.07 |
| | dimethicone⁵ | | | | | | | |
| C | Iron Oxide (CI 77491), | 0.25 | 0.25 | 0.25 | 0.25 | 0.25 | 0.25 | 0.25 |
| | dimethicone⁶ | | | | | | | |
| C | Iron Oxide (CI 77492), | 1.09 | 1.09 | 1.09 | 1.09 | 1.09 | 1.09 | 1.09 |
| | dimethicone⁷ | | | | | | | |
| C | Titanium Dioxide (and) | 5.81 | 5.81 | 5.81 | 5.81 | 5.81 | 5.81 | 5.81 |
| | dimethicone⁸ | | | | | | | |
| C | Caprylyl Methicone² | 3.28 | 3.28 | 3.28 | 3.28 | 3.28 | 3.28 | 3.28 |
| D | Isododecane (and) Acrylates/ | — | 1.5 | — | — | — | — | — |
| | Polytimethylsiloxymethacrylate | | | | | | | |
| | Copolymer⁹ | | | | | | | |
| D | Example S8 | — | — | 0.6 | — | — | — | — |
| D | Example S14 | — | — | — | 0.6 | — | — | — |
| D | Example S15 | — | — | — | — | 0.6 | — | — |
| D | Example S16 | — | — | — | — | — | 0.6 | — |
| D | Example S17 | — | — | — | — | — | — | 0.6 |

¹Available from The Dow Chemical Company under tradename DOWSIL ™ ES-5300.
²Available from The Dow Chemical Company under tradename DOWSIL ™ FZ-3196.
³Available from The Innovation Company under tradename Creasil ID CG.
⁴Available from Schulke Inc. under tradename Euxyl PE 9010.
⁵Available from Miyoshi America under tradename SAT-B-335198.
⁶Available from Miyoshi America under tradename SAT-Y-338075.
⁷Available from Miyoshi America under tradename SAT-R-338073.
⁸Available from Miyoshi America under tradename SAT-TRI-77891.
⁹Available from The Dow Chemical Company under tradename DOWSIL ™ FA4002 ID.

### Wear Resistance

The skin care formulations prepared according to Comparative Examples C₂-C₃ and Examples 5-9 were each coated on a white vinyl chart (available from Leneta) using a doctor blade film applicator with the gap set at 6 mils (0.1524 mm) and allowed to dry at 22° C. for at least 48 hours. The color reading of each sample was then measured by colorimeter (Ocean Optics). The wear resistance of the deposited film of skin care formulations was characterized by the change (ΔE) before and after abrasion with a pre-cut bath towel (55 mm×45 mm). The bath towel was fixed to a moving robotic part that moves back and forth periodically at a constant speed. The film was abraded by the bath towel by 3 and 6 wear cycles under a pressure of approximately 600 Pa, each wear cycle lasts 6 seconds. Readings were taken from ten point on each deposited film. The average of the readings from each sample is provided in TABLE 7. Lower ΔE indicates less color change after rub-off and higher wear resistance. The ΔE of the inventive polymers were lower than the commercial material, which is highly favorable for skin care formulations to enable long lasting performance.

TABLE 7

Wear Cycles

| | 3 | 6 |
|---|---|---|
| Skin care formulation | ΔE | ΔE |
| Comp. Example C2 | 10.51 | 12.03 |
| Comp. Example C3 | 7.86 | 11.44 |
| Example 5 | 5.70 | 7.85 |
| Example 6 | 4.96 | 7.40 |
| Example 7 | 8.07 | 10.93 |
| Example 8 | 5.60 | 8.62 |
| Example 9 | 7.15 | 8.47 |

### Comparative Examples C4-C13 and Examples 10-28: Viscosity

The viscosity of oils alone, Comparative Examples C4-C13, and thickening oil/polymer blends, Examples 10-28, as noted in TABLE 8 was determined using a TA Instruments DHR-3 rheometer at 25° C., equipped with a stainless steel 60 mm, 0.5° cone and plate sensor, a gap set at 17 microns and a shear rate of 0.01 s⁻¹. The viscosities measured are reported in TABLE 8.

TABLE 8

| | | Thickening | Conc. TP | |
|---|---|---|---|---|
| | | Polymer | in Oil | Viscosity |
| Example | Oil | (“TP”) | (wt %) | (mPa · s) |
| C4 | XIAMETER ™ PMX-200 | — | — | 4.81 × 10⁻¹ |
| | Silicone Fluid, 0.65 cSt^{a} | | | |
| C5 | Cetiol Ultimate^{b} | — | — | 1.20 × 10⁰ |
| C6 | Isododecane | — | — | 1.22 × 10⁰ |
| C7 | 2-methylundecenoate | — | — | 1.89 × 10⁰ |
| C8 | Octamethylcyclotetrasiloxane | — | — | 2.24 × 10⁰ |
| C9 | Hemisqualane^{c} | — | — | 2.42 × 10⁰ |
| C10 | XIAMETER ™ PMX-200 | — | — | 4.60 × 10⁰ |
| | Silicone Fluid, 5 cSt^{d} | | | |
| C11 | 2-butyl-1-octanole | — | — | 1.91 × 10² |
| C12 | DOWSIL ™ 4-2737 Fluid^{f} | — | — | 4.47 × 10¹ |
| C13 | Sunflower Oil | — | — | 1.55 × 10² |
| 10 | XIAMETER ™ PMX-200 | Example S2 | 2.5 | 2.38 × 10² |
| | Silicone Fluid, 0.65 cSt^{a} | | | |
| 11 | Cetiol Ultimate^{b} | Example S2 | 2.5 | 9.49 × 10² |
| 12 | Isododecane | Example S2 | 2.5 | 3.91 × 10² |
| 13 | 2-methylundecenoate | Example S2 | 2.5 | 1.81 × 10³ |
| 14 | Octamethylcyclotetrasiloxane | Example S2 | 2.5 | 3.14 × 10³ |
| 15 | Hemisqualane^{c} | Example S2 | 2.5 | 1.63 × 10³ |
| 16 | XIAMETER ™ MPX-200 | Example S2 | 2.5 | 4.97 × 10² |
| | Silicone Fluid, 5 cSt^{d} | | | |
| 17 | 2-butyl-1-octanol^{e} | Example S2 | 2.5 | Gel |
| 18 | DOWSIL ™ 4-2737 Fluid^{f} | Example S2 | 2.5 | B |
| 19 | Sunflower Oil | Example S2 | 2.5 | B |
| 20 | Cetiol Ultimate^{b} | Example S2 | 5.0 | 4.73 × 10⁴ |
| 21 | Isododecane | Example S2 | 5.0 | 4.10 × 10⁴ |
| 22 | 2-methylundecenoate | Example S2 | 5.0 | 5.33 × 10⁴ |
| 23 | Hemisqualane^{c} | Example S2 | 5.0 | 2.25 × 10⁴ |
| 24 | Cetiol Ultimate^{b} | Example S1 | 8.0 | 4.35 × 10⁴ |
| 25 | Hemisqualane^{c} | Example S1 | 8.0 | 1.19 × 10⁵ |
| 26 | Isododecane | Example S1 | 8.0 | 3.30 × 10⁴ |
| 27 | 2-butyl-1-octanol^{e} | Example S1 | 8.0 | 1.52 × 10⁵ |
| 28 | 2-methylundecenoate | Example S1 | 8.0 | 8.34 × 10³ |

^{a}avalible from The Dow Chemical Company
^{b}INCI: Undecane (and) tridecane avaIlable from BASF
^{c}Fully saturated C₁₅ hydrocarbons (99%) available from Sigma-Aldrich
^{d}available from The Dow Chemical Company
^{e}Isofol 12 available from Sasol
^{f}Linear siloxane fluid available from The Dow Chemical Company
B - inhomogeneous with suspended chunks

We claim:

1. An oil/polymer blend comprising:
(a) an oil, wherein the oil is selected from the group consisting of silicone fluids, fluids containing 2 to 30 carbon atoms per molecule, and mixtures thereof;
(b) a thickening polymer powder, thickening polymer, wherein the thickening polymer is a silylated cellulose polymer, comprising a cellulose polymer functionalized with —Si(R¹)₃ groups; wherein each R¹ is independently selected from the group consisting of a hydrogen, a C₁₋₈ alkyl group, a C₁₋₈ haloalkyl group, an aryl group, a C₁₋₈ alkylaryl group and a C₁₋₈ haloalkylaryl group;
wherein the cellulose polymer has a weight average molecular weight of 50,000 to 1,500,000 Daltons.

2. The oil/polymer blend of claim 1, wherein the oil is a cosmetically acceptable oil.

3. The oil/polymer blend of claim 2, wherein the cosmetically acceptable oil is selected from the group consisting of linear or branched C₈₋₃₀ alkanes; C₈₋₃₀ alkyl esters; C₈₋₃₀ carboxylic acid esters; C₈₋₃₀ linear or branched alcohols; silicone fluids; and mixtures thereof.

4. The oil/polymer blend of claim 1, wherein the silylated cellulose polymer is of formula I 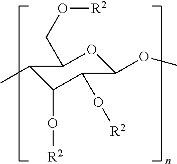
wherein n is an average of 308 to 9,252; wherein each R² is independently selected from the group consisting of a hydrogen, a C₁₋₈ alkyl group and a —Si(R¹)₃ group; wherein each R¹ is independently selected from the group consisting of a hydrogen, a C₁₋₈ alkyl group, a C₁₋₈ haloalkyl group, an aryl group, a C₁₋₈ alkylaryl group and a C₁₋₈ haloalkylaryl group.

5. The oil/polymer blend of claim 3, further comprising a cosmetically acceptable carrier and a color ingredient; and wherein the oil/polymer blend is a skin care formulation.

6. The skin care formulation of claim 5, wherein the color ingredient is selected from the group consisting of inorganic pigments, organic pigments, dyes, aqueous pigment dispersions and mixtures thereof.

7. The skin care formulation of claim 5, wherein the color ingredient is selected from the group consisting of Ext. D&C Yellow No. 2, Ext. D & C Violet No. 2, FD&C Red No. 4, FD&C Red No. 40, FD&C Yellow No. 5, FD&C Yellow No. 6, FD&C Green No. 3, FD&C Blue No. 1, D&C Yellow No. 7, D&C Yellow No. 8, D&C Yellow No. 10, D&C Yellow No. 11, D&C Violet No. 2, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 34, D&C Red No. 33, D&C Red No. 36, D&C Green No. 5, D&C Green No. 6, D&C Green No. 8, D&C Blue No. 4, D&C Orange No. 4, D&C Orange No. 5, D&C Orange No. 10, D&C Orange No. 11, D&C Brown No. 1, Aluminum powder, Annatto, Bismuth citrate, Bismuth Oxychloride, Bronze powder, Caramel, Carmine, 0-Carotene, Chromium hydroxide green, Chromium oxide green, Copper chlorophyllin, Copper powder, Dihydroxyacetone, Ferric Ammonium ferrocyanide, Ferric ferrocyanide, Guanine, Iron oxide, Manganese Violet, Mica, Silver, Titanium Dioxide, Ultramarine, Zinc Oxide and mixtures thereof.

8. The skin care formulation of claim 5, wherein the color ingredient includes an iron oxide and titanium dioxide.

9. The skin care formulation of claim 4, further comprising a suncare active.

10. A method of enhancing the appearance of skin, comprising:
providing a skin care formulation of claim 5, and
applying the skin care formulation to skin.
